(19) United States
(12) Patent Application Publication
(10) Pub. No.: US 2026/0294292 A1
(43) Pub. Date: Oct. 1, 2026

(54) SENSOR FOR ANALYTE MONITORING WITH DRUG-ELUTING CAPABILITIES

(71) Applicant: Senseonics, Incorporated, Germantown, MD (US)

(72) Inventors: James Masciotti, Germantown, MD (US); Philip Huffstetler, Germantown, MD (US); Venkata Velvadapu, Germantown, MD (US); Giacomo Fornasini, Germantown, MD (US)

(73) Assignee: Senseonics, Incorporated, Germantown, MD (US)

(21) Appl. No.: 19/554,852

(22) Filed: Mar. 3, 2026

Related U.S. Application Data
(60) Provisional application No. 63/780,935, filed on Mar. 31, 2025.

(51) Int. Cl. A61B 5/1473; A61B 5/00; A61K 9/00; A61K 31/192; A61K 31/573; A61K 31/616

(52) U.S. Cl. CPC A61B 5/14735; A61B 5/6861; A61K 9/0024; A61K 31/192; A61K 31/573; A61K 31/616; A61B 2562/12

(57) ABSTRACT

A sensor (e.g., an analyte sensor) that may be implanted partially or fully within a living animal (e.g., a human) and may be used to measure an analyte (e.g., glucose or oxygen) in a medium (e.g., interstitial fluid, blood, or intraperitoneal fluid) within the animal. The sensor may include a housing including one or more indicator portions, a plurality of drug portions on opposite sides of each indicator portion, circuitry within the housing, including a sensing area, analyte indicator material, and a plurality of drug-eluting material in or on the drug portions of the exterior surface of the housing. The drug-eluting material each comprise one or more therapeutic agents that reduce deterioration of the analyte indicator material.

### CROSS-REFERENCE TO RELATED APPLICATION

The present application claims the benefit of priority to U.S. Provisional Application Ser. No. 63/780,935, filed on Mar. 31, 2025, which is incorporated herein by reference in its entirety.

### BACKGROUND

### Field of Invention

The present invention relates generally to analyte monitoring. More specifically, the present invention relates to a sensor including a housing comprising one or more indicator portions and drug-eluting portions, analyte indicator material for analyte detection on or in the one or more indicator portions, and drug-eluting material that release one or more therapeutic agents that reduce deterioration of the analyte indicator material on or in the drug-eluting portions.

### Discussion of the Background

A sensor may be implanted (partially or fully) within a living animal (e.g., a human) and used to measure an analyte (e.g., glucose, oxygen, cardiac markers, low-density lipoprotein (LDL), high-density lipoprotein (HDL), or triglycerides) in a medium (e.g., interstitial fluid (ISF), blood, or intraperitoneal fluid) within the living animal. The sensor may include a light source (e.g., a light-emitting diode (LED) or other light emitting element), indicator molecules, and a photodetector (e.g., a photodiode, phototransistor, photoresistor or other photosensitive element). Examples of implantable sensors employing indicator molecules to measure an analyte are described in U.S. Pat. Nos. 5,517,313 and 5,512,246, which are incorporated herein by reference in their entirety.

A sensor may include analyte indicator material, which may be include indicator molecules. For example, in an implantable fluorescence-based glucose sensor, fluorescent indicator molecules may reversibly bind glucose and, when irradiated with excitation light (e.g., light having a wavelength of approximately 378 nm), each indicator molecule may emit an amount of light (e.g., light in the range of 400 to 500 nm) that depends on whether glucose is bound to the indicator molecule.

If a sensor or other medical device is implanted in the body of a living animal, the animal's immune system may begin to attack the sensor or medical device. For instance, if a sensor or other medical device is implanted in a human, white blood cells may attack the sensor or other medical device as a foreign body, and, in the initial immune system onslaught, neutrophils may be the primary white blood cells attacking the sensor. The defense mechanism of neutrophils includes the release of highly caustic substances known as reactive oxygen and nitrogen species. The reactive oxygen species include, for example, hydrogen peroxide.

Hydrogen peroxide and other reactive species such as reactive oxygen and nitrogen species may degrade the indicator molecules of an analyte indicator. For instance, in indicator molecules having a boronate group, hydrogen peroxide may degrade the indicator molecules by oxidizing the boronate group, thus disabling the ability of the indicator molecule to bind glucose. In addition, such reactive species degrade ester-containing polymers of an analyte indicator, for example, as described by Reid, B. et al. PEG hydrogel degradation and the role of the surrounding tissue environment. J. of Tissue Engr, and Regen. Med. 2015.

Glucocorticoids are used with cardiac pace makers and eye surgery to reduce inflammation. For instance, the following European patent application publications describe pace-maker leads and controlled release of steroids: EP2416783 A1 (“Improved glucocorticoid therapy”), EP1477187 B1 (“Formulation for controlled release of drugs by combining hydrophilic and hydrophobic agents”), EP1637164 A2 (“Improved formulation for controlled release of drugs by combining hydrophilic and hydrophobic agents”), and EP2303227 A2 (“Controlled release corticosteroid compositions and methods for the treatment of optic disorders”). However, these devices do not have analyte indicators, and the glucocorticoid is not used to reduce degradation of an analyte indicator. Instead, the glucocorticoid is used to stop scar tissue from building up.

There is presently a need in the art for improvements in reducing analyte indicator degradation.

### SUMMARY

The present invention overcomes the disadvantages of prior systems by providing, among other advantages, reduced analyte indicator degradation.

One aspect of the present invention may provide a sensor for measurement of an analyte in a medium within a living animal. The sensor may include a housing, circuitry within the housing, analyte indicator material, a first drug-eluting material, and a second drug-eluting material. The housing may include an exterior surface. The exterior surface of the housing may include an indicator portion, a first drug portion on a first side of the indicator portion, and a second drug portion on a second side of the indicator portion that is opposite the first side of the indicator portion. The circuitry may include a sensing area that includes one or more light sources and one or more photodetectors. The indicator portion of the housing may correspond to the sensing area of the circuitry. The analyte indicator material may be on or in only the indicator portion of the exterior surface of the housing. The analyte indicator material may include indicator molecules configured to reversibly bind the analyte, and the indicator molecules may have a detectable property indicative of whether the analyte is bound or unbound. The one or more lights sources of the sensing area may be configured to emit light to the analyte indicator material, and the one or more photodetectors of the sensing area may be configured to receive light reflected from or emitted by the analyte indicator material. The first drug-eluting material may be in or on the first drug portion of the exterior surface of the housing. The second drug-eluting material may be in or on the second drug portion of the exterior surface of the housing. The first and second drug-eluting material may each include one or more therapeutic agents that reduce deterioration of the analyte indicator material.

In some aspects, the sensor may be implantable within a living animal.

In some aspects, the analyte indicator material may not extend, in a direction of a longitudinal axis of the housing, beyond the sensing area of the circuitry.

In some aspects, the indicator portion of the housing may be a first indicator portion, the sensing area may be a first sensing area, and the analyte indicator material may be a first analyte indicator material. In such aspects, the exterior surface of the housing may further include a second indicator portion, a third drug portion on a first side of the second indicator portion, and a fourth drug portion on a second side of the second indicator portion that is opposite the first side of the second indicator portion. In such aspects, the circuitry may further include a second sensing area that includes one or more light sources and one or more photodetectors, and the second indicator portion of the housing may correspond to the second sensing area of the circuitry. In such aspects, the sensor may further include second analyte indicator material on or in only the second indicator portion of the exterior surface of the housing, the second analyte indicator material may include indicator molecules configured to reversibly bind the analyte, and the indicator molecules of the second analyte indicator material may have a detectable property indicative of whether the analyte is bound or unbound. In such aspects, the one or more lights sources of the second sensing area may be configured to emit light to the second analyte indicator material. In such aspects, the one or more photodetectors of the second sensing area may be configured to receive light reflected from or emitted by the second analyte indicator material. In such aspects, the third drug-eluting material may be in or on the third drug portion of the exterior surface of the housing, and the fourth drug-eluting material may be in or on the fourth drug portion of the exterior surface of the housing. The third and fourth drug-eluting material may each include one or more therapeutic agents that reduce deterioration of at least the second analyte indicator material.

In some aspects, the one or more therapeutic agents of the first and second drug-eluting material may be incorporated within the first and second drug-eluting material via one or more covalent bonds that break in the presence of aqueous media and release the one or more therapeutic agents of the first and second drug-eluting material. In some alternative aspects, the one or more of the therapeutic agents of the first and second drug-eluting material may be incorporated within the first and second drug-eluting material via one or more covalent bonds that break through exposure to light and release the one or more therapeutic agents of the first and second drug-eluting material.

In some aspects, the one or more therapeutic agents of the first and second drug-eluting material may include an anti-inflammatory drug. In some aspects, the anti-inflammatory drug may be a non-steroidal anti-inflammatory drug. In some aspects, the non-steroidal anti-inflammatory drug may be acetylsalicylic acid. In some aspects, the non-steroidal anti-inflammatory drug may be isobutylphenylpropanoic acid.

In some aspects, the one or more therapeutic agents of the first and second drug-eluting material may include a glucocorticoid. In some aspects, the one or more therapeutic agents of the first and second drug-eluting material may include one or more of dexamethasone, triamcinolone, betamethasone, methylprednisolone, beclometasone, fludrocortisone, derivatives thereof, and analogs thereof.

In some aspects, the one or more therapeutic agents of the first and second drug-eluting material may reduce oxidation of the analyte indicator.

Another aspect of the present invention may provide a method of manufacturing a sensor for measurement of an analyte in a medium within a living animal. The method may include providing first drug-eluting material in or on a first drug portion of an exterior surface of a housing of the sensor. The exterior surface of the housing may include an indicator portion, the first drug portion on a first side of the indicator portion, and a second drug portion on a second side of the indicator portion that is opposite the first side of the indicator portion. The sensor may include circuitry within the housing, the circuitry may include a sensing area that includes one or more light sources and one or more photodetectors, and the indicator portion of the housing may correspond to the sensing area of the circuitry. The sensor may include analyte indicator material on or in only the indicator portion of the exterior surface of the housing. The analyte indicator material may include indicator molecules configured to reversibly bind the analyte, and the indicator molecules may have a detectable property indicative of whether the analyte is bound or unbound. The one or more lights sources of the sensing area may be configured to emit light to the analyte indicator material. The one or more photodetectors of the sensing area may be configured to receive light reflected from or emitted by the analyte indicator material. The method may include providing second drug-eluting material in or on the second drug portion of the exterior surface of the housing. The first and second drug-eluting material may each include one or more therapeutic agents that reduce deterioration of the analyte indicator material.

In some aspects, the sensor may be implantable within a living animal. In some aspects, the analyte indicator material may not extend, in a direction of a longitudinal axis of the housing, beyond the sensing area of the circuitry.

In some aspects, the indicator portion of the housing may be a first indicator portion, the sensing area may be a first sensing area, and the analyte indicator material may be first analyte indicator material. In some aspects, the exterior surface of the housing may further include a second indicator portion, a third drug portion on a first side of the second indicator portion, and a fourth drug portion on a second side of the second indicator portion that is opposite the first side of the second indicator portion. In some aspects, the circuitry may further include a second sensing area that includes one or more light sources and one or more photodetectors, and the second indicator portion of the housing may correspond to the second sensing area of the circuitry. In some aspects, the sensor may further include second analyte indicator material on or in only the second indicator portion of the exterior surface of the housing. In some aspects, the second analyte indicator material may include indicator molecules configured to reversibly bind the analyte, and the indicator molecules of the second analyte indicator material may have a detectable property indicative of whether the analyte is bound or unbound. In some aspects, the one or more lights sources of the second sensing area may be configured to emit light to the second analyte indicator material. The one or more photodetectors of the second sensing area may be configured to receive light reflected from or emitted by the second analyte indicator material. In some aspects, the method may further include providing third drug-eluting material in or on the third drug portion of the exterior surface of the housing. In some aspects, the method may further include providing fourth drug-eluting material in or on the fourth drug portion of the exterior surface of the housing. In some aspects, the third and fourth drug-eluting material may each include one or more therapeutic agents that reduce deterioration of at least the second analyte indicator material. In some aspects, the first, second, third, and fourth drug-eluting material may be pieces of an initial drug-eluting material, and the method may further include dividing the initial drug-eluting material into pieces to create the first, second, third, and fourth drug-eluting material.

In some aspects, the one or more therapeutic agents of the first and second drug-eluting material may be incorporated within the first and second drug-eluting material via one or more covalent bonds that break in the presence of aqueous media and release the one or more therapeutic agents of the first and second drug-eluting material. In some alternative aspects, the one or more of the therapeutic agents of the first and second drug-eluting material may be incorporated within the first and second drug-eluting material via one or more covalent bonds that break through exposure to light and release the one or more therapeutic agents of the first and second drug-eluting material.

In some aspects, the one or more therapeutic agents of the first and second drug-eluting material may include an anti-inflammatory drug. In some aspects, the anti-inflammatory drug may be a non-steroidal anti-inflammatory drug. In some aspects, the one or more therapeutic agents of the first and second drug-eluting material may include a glucocorticoid. In some aspects, the one or more therapeutic agents of the first and second drug-eluting material may include one or more of dexamethasone, triamcinolone, betamethasone, methylprednisolone, beclometasone, fludrocortisone, derivatives thereof, and analogs thereof. In some aspects, the one or more therapeutic agents of the first and second drug-eluting material may reduce oxidation of the analyte indicator.

In some aspects, the first drug-eluting material and the second drug-eluting material may be pieces of an initial drug-eluting material, and the method may further include dividing the initial drug-eluting material into pieces to create the first drug-eluting material and the second drug-eluting material.

Further variations encompassed within the invention are described in the detailed description of the invention below.

### BRIEF DESCRIPTION OF THE DRAWINGS

The accompanying drawings, which are incorporated herein and form part of the specification, illustrate various, non-limiting embodiments of the present invention. In the drawings, like reference numbers indicate identical or functionally similar elements.

FIG. 1 is a schematic view illustrating a system embodying aspects of the present invention.

FIG. 2 is a schematic view illustrating a system embodying aspects of the present invention.

FIGS. 3A and 3B illustrate an exploded perspective view and an assembled top view, respectively, of a sensor of the system according to some aspects.

FIG. 4 is a top view of a sensor area of circuitry of the sensor according to some aspects of the present invention.

FIGS. 5A and 5B show top views illustrating sensors embodying aspects of the present invention. In FIG. 5B, the first drug-eluting material and second drug-eluting material are closer together than the first and second drug-eluting material of FIG. 5A.

FIGS. 6A and 6B show side views illustrating sensors embodying aspects of the present invention. The distances between the indicator and portions of the housing of the sensors are varied between FIG. 6A and FIG. 6B.

FIGS. 7A-7D show burst oxidation of analyte indicator material of a sensor according to some aspects of the present invention, including burst oxidation of a long end distal portion of analyte indicator material on or in a first indicator portion of a housing of the sensor (FIG. 7A), burst oxidation of a long end central portion of the analyte indicator material on or in the first indicator portion of the housing (FIG. 7B), burst oxidation of a short end central portion of analyte indicator material on or in a second indicator portion of the housing (FIG. 7C), and burst oxidation of a short end distal portion of the analyte indicator material on or in the second indicator portion of the housing (FIG. 7D).

FIGS. 8A-8D show burst oxidation of analyte indicator material of a sensor according to some aspects of the present invention, including burst oxidation of a long end distal portion of analyte indicator material on or in a first indicator portion of a housing of the sensor (FIG. 8A), burst oxidation of a long end central portion of the analyte indicator material on or in the first indicator portion of the housing (FIG. 8B), burst oxidation of a short end central portion of analyte indicator material on or in a second indicator portion of the housing (FIG. 8C), and burst oxidation of a short end distal portion of the analyte indicator material on or in the second indicator portion of the housing (FIG. 8D).

FIGS. 9A-9D show burst oxidation of analyte indicator material of a sensor according to some aspects of the present invention, including burst oxidation of a long end distal portion of analyte indicator material on or in a first indicator portion of a housing of the sensor (FIG. 9A), burst oxidation of a long end central portion of the analyte indicator material on or in the first indicator portion of the housing (FIG. 9B), burst oxidation of a short end central portion of analyte indicator material on or in a second indicator portion of the housing (FIG. 9C), and burst oxidation of a short end distal portion of the analyte indicator material on or in the second indicator portion of the housing (FIG. 9D).

FIG. 10 shows responsivity of a sensor over time according to some aspects.

FIG. 11 is a flowchart illustrating an example of a process for manufacturing a sensor embodying aspects of the present invention.

### DETAILED DESCRIPTION

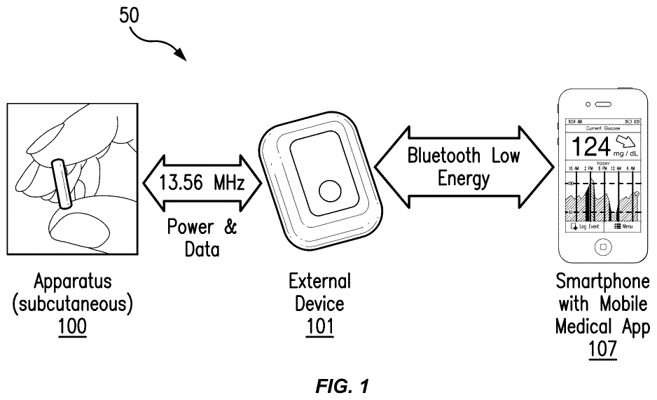

FIG. 1 is a schematic view of an exemplary system 50 embodying aspects of the present invention. In some aspects, the system 50 may be an analyte monitoring system. In some aspects, the system 50 may be a continuous analyte monitoring system (e.g., a continuous glucose monitoring system). In some aspects, the system 50 may include a sensor 100, an external device 101, and/or a display device 107.

In some aspects, the sensor 100 may be an implantable device. In some aspects, the sensor 100 may be a wireless implantable device. In some aspects, the sensor 100 may be a specific sensor (e.g., an analyte sensor). In some aspects, the sensor 100 may include one or more optical sensors (e.g., one or more fluorometers). In some aspects, the sensor 100 may include one or more chemical or biochemical sensors. In some aspects, the sensor 100 may be a radio frequency identification (RFID) device. In some aspects, the sensor 100 may be a small, fully subcutaneously implantable sensor that detects the presence, amount, and/or concentration of an analyte (e.g., glucose, oxygen, cardiac markers, low-density lipoprotein (LDL), high-density lipoprotein (HDL), or triglycerides) in a medium (e.g., interstitial fluid) of a living animal (e.g., a living human). However, this is not required, and, in some alternative aspects, the sensor 100 may be a partially implantable (e.g., transcutaneous) device or a fully external sensor.

In some aspects, the external device 101 may be an externally worn device (e.g., attached via an armband, wristband, waistband, or adhesive patch). In some aspects, the external device 101 may remotely communicate with the sensor 100 (e.g., via near field communication (NFC) or far field communication). In some aspects, the external device 101 may communicate with the sensor 100 to initiate and/or read data (e.g., measurements) from the sensor 100. In some aspects, the external device 101 may be a transceiver. In some aspects, the external device 101 may be a smartphone (e.g., an NFC-enabled and/or Bluetooth Low Energy (BLE)-enabled smartphone). In some aspects, the external device 101 may communicate information (e.g., one or more analyte measurements) wirelessly (e.g., via a Bluetooth™ communication standard such as, for example and without limitation Bluetooth Low Energy) to an application running on a display device 107 (e.g., smartphone). In some aspects, the display device 107 may additionally or alternatively communicate directly with the sensor 100 (e.g., via near field communication (NFC)). In some aspects, the display device 107 may communicate with the sensor 100 to initiate and/or read data (e.g., measurements) from the sensor 100.

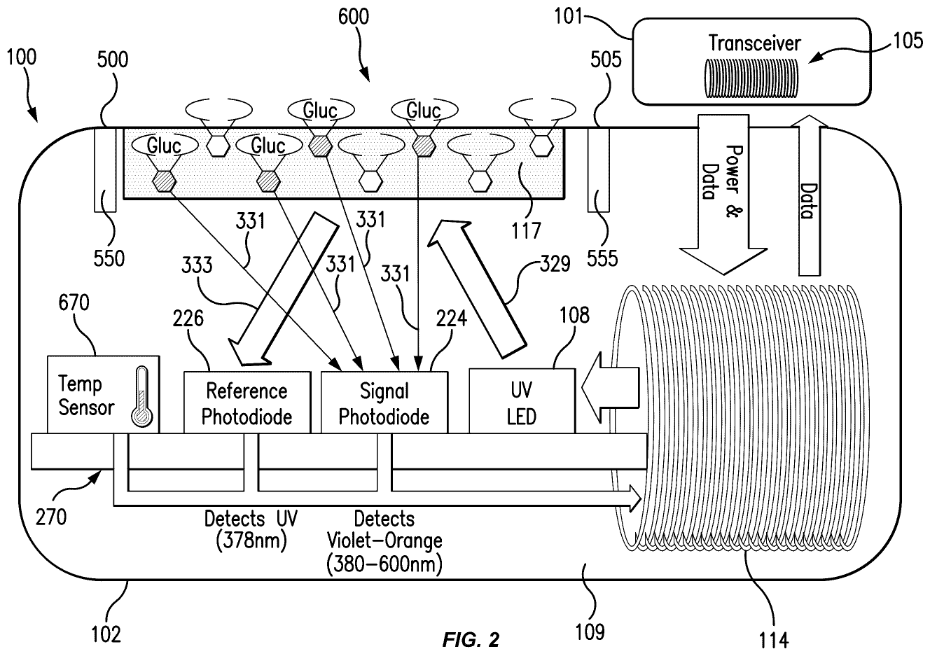

In some aspects, the external device 101 may be an electronic device that communicates with the sensor 100 to power the sensor 100, provide commands and/or data to the sensor 100, and/or receive data from the sensor 100. For example, in some aspects, as shown in FIG. 2, the external device 101 may include an antenna 105 (e.g., an inductor such as a coil), and the external device 101 may convey data by modulating the electromagnetic wave generated by the antenna 105 (e.g., by modulating the current flowing through the antenna 105 of the external device 101). In some aspects, the received data may include one or more sensor measurements. In some aspects, the sensor measurements may include, for example and without limitation, one or more light measurements from one or more photodetectors of the sensor 100 and/or one or more temperature measurements from one or more temperature sensors 670 of the sensor 100. In some aspects, the external device 101 may receive data by detecting modulations in the electromagnetic wave generated by the sensor 100, e.g., by detecting modulations in the current flowing through the antenna of the external device 101. In some aspects, the external device 101 may calculate analyte (e.g., glucose) concentrations measurement information conveyed by the sensor 100.

In some aspects, the external device 101 may use the antenna 105 to generate an electromagnetic wave or electrodynamic field to induce a current in an antenna 114 (e.g., inductor) of the sensor 100. In some aspects, the sensor 100 may use the current induced in the antenna 114 to power the sensor 100. However, this is not required, and, in some alternative aspects, the sensor 100 may be powered by an internal power source (e.g., a battery).

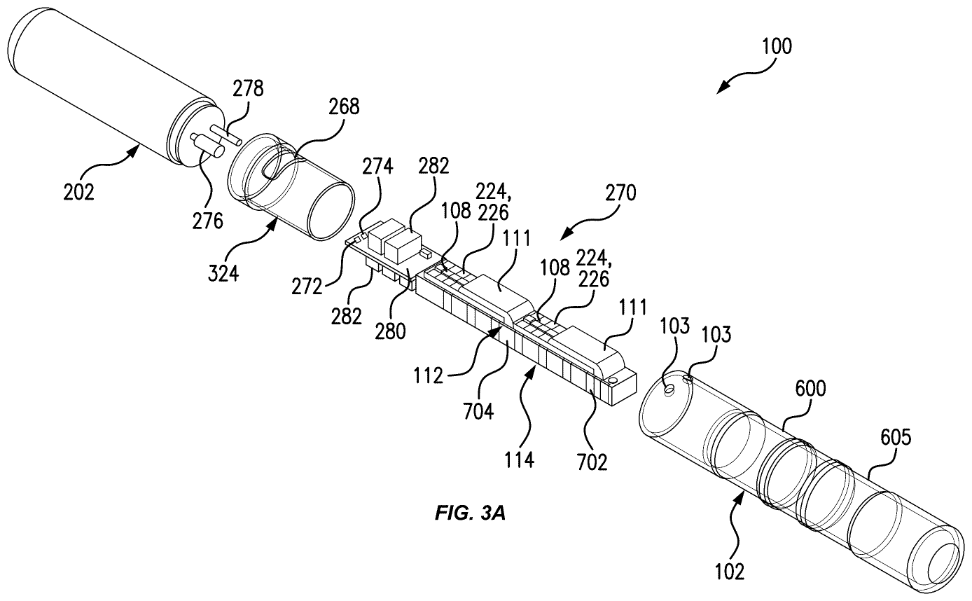

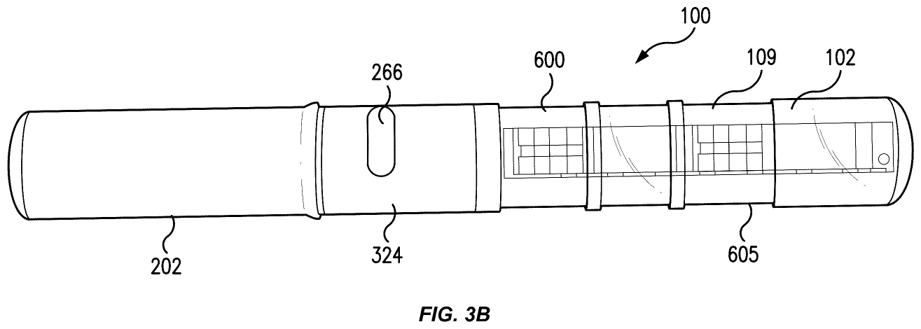

In some aspects, as shown in FIGS. 2, 3A, 3B, 5A, 5B, 6A, and 6B, the sensor 100 may include a housing 102 (e.g., body, shell, capsule, or encasement), which may be rigid and biocompatible. In some aspects, the housing 102 may be formed from a suitable, optically transmissive polymer material, such as, for example, acrylic polymers (e.g., polymethylmethacrylate (PMMA)). In some aspects, the exterior surface of the housing 102 may include one or more indicator portions 600, 605. For example, in some single indicator portion aspects, as shown in FIG. 2, the exterior surface of the housing 102 of the sensor 100 may include a single indicator portion 600. For another example, in some aspects with multiple indicator portions, as shown in FIGS. 3A, 3B, 5A, 5B, 6A, and 6B, the indicator portion 600 may be a first indicator portion, and the exterior surface of the housing 102 of the sensor 100 may additionally include at least a second indicator portion 605. In some aspects with multiple indicator portions, the analyte indicator material 117 of the first indicator portion 600 may be the same as the analyte indicator material 117 of the second indicator portion 605. However, this is not required, and, in some alternative aspects with multiple indicator portions, the analyte indicator material 117 of the first indicator portion 600 may be different than the analyte indicator material 117 of the second indicator portion 605.

In some aspects, as shown in FIG. 2, the sensor 100 may include analyte indicator material 117 on or in only the one or more indicator portions 600, 605 of the exterior surface of the housing 102. In some aspects, the analyte indicator material 117 on or in indicator portions 600, 605 of the housing 102 may be porous and may allow an analyte (e.g., glucose) in a medium (e.g., interstitial fluid) to diffuse into the analyte indicator material 117. In some aspects, as shown in FIG. 2, the analyte indicator material 117 may include indicator molecules 104 configured to reversibly bind the analyte. In some aspects, the analyte indicator material 117 may be, for example, a polymer graft or hydrogel coated, diffused, adhered, embedded, or grown on or in the indicator portion 600 of the exterior surface of the housing 102. In some aspects, the indicator molecules 104 may be distributed throughout the analyte indicator material 117. In some aspects, the indicator molecules 104 may have one or more detectable properties (e.g., optical properties) that vary in accordance with the amount or concentration of an analyte in proximity to the analyte indicator material 117. In some aspects, the indicator molecules 104 may be, for example, fluorescent analyte indicator molecules. In some aspects, the indicator molecules 104 may be phenylboronic-based analyte indicator molecules. However, phenylboronic-based analyte indicator molecules are not required, and, in some alternative aspects, the analyte indicator material 117 may include different indicator molecules 104, such as, for example and without limitation, glucose oxidase-based indicator molecules, glucose dehydrogenase-based indicator molecules, and glucose binding protein-based indicators. In some aspects, the indicator molecules 104 in the analyte indicator material 117 may be selected from a group including fluorescent indicator molecules (e.g., TFM. having the chemical name 9-[N-[6-(4,4,5,5,-tetramethyl-1,3,2-dioxaborolano)-3-(trifluoromethyl)benzyl]-N-[3-(methacrylamido) propylamino]methyl]-10-[N-[6-(4,4,5,5,-tetramethyl-1,3,2-dioxaborolano)-3-(trifluoromethyl)benzyl]-N-[2-(carboxyethyl)amino]methyl]anthracene sodium salt) or light absorbing, non-fluorescent indicator molecules. In some aspects, the indicator molecules 104 may have a detectable property indicative of whether the analyte is bound or unbound.

In some aspects, as shown in FIGS. 2 and 3A, the sensor 100 may include circuitry 270 in the housing 102. In some aspects, the circuitry 270 may include the antenna 114. In some aspects, the circuitry 270 may include one or more sensing areas. In some aspects, as shown in FIGS. 2, 3A, 4, 5A, 5B, 6A, and 6B, a sensing area of the circuitry 270 may include one or more light sources 108 and one or more photodetectors 224, 226 that interact with analyte indicator material 117 in or on an indicator portion 600 or 605 of the housing 102. In some aspects, as shown in FIG. 2, a sensing area of the circuitry 270 may include one or more temperature sensors 670. In some aspects, the number of sensing areas of the circuitry 270 may correspond to the number of indicator portions 600, 605 of the housing 102 (e.g., circuitry 270 may include one sensing area per indicator portion, two sensing areas per indicator portion, or three sensing areas per indicator portion, etc.). For example, in some single indicator portion aspects, as shown in FIG. 2, the circuitry 270 may include a single sensing area that interacts with the analyte indicator material 117 in or on the single indicator portion 600 of the exterior surface of the housing 102. For another example, in some multiple indicator portions aspects, as shown in FIGS. 3A, 3B, 5A, 5B, 6A, and 6B, the circuitry 270 may include first and second sensing areas that interact with the analyte indicator material 117 in or on the first and second indicator portions 600 and 605, respectively, of the exterior surface of the housing 102.

In some aspects, the one or more light sources 108 of a sensing area of the circuitry 270 may be, for example, one or more light emitting diodes (LEDs) or other light sources that emit radiation, including radiation over a range of wavelengths that interact with the indicator molecules 104. In some aspects, as shown in FIG. 2, a light source 108 may emit the excitation light 329 that irradiates the indicator molecules 104 in the analyte indicator material 117 of an indicator portion 600 of the housing 102. In some aspects, the light source 108 may emit excitation light 329, for example, at a wavelength of approximately 378 nm. However, this is not required, and, in some alternative aspects, the light source 108 may emit excitation light 329 at a different wavelength.

In some aspects, the one or more photodetectors of a sensing area of the circuitry 270 may be, for example, photodiodes, phototransistors, photoresistors or other photosensitive elements. In some aspects, as illustrated in FIGS. 2, 3A, 4, 5A, 5B, 6A, and 6B, a sensing area of the circuitry 270 may include one or more first photodetectors 224 and one or more second photodetectors 226. However, this is not required, and, in some alternative aspects, a sensing area of the circuitry 270 may only include the one or more first photodetectors 224. In the case of a fluorescence-based sensor, the one or more first photodetectors 224 may be sensitive to fluorescent light 331 emitted by the indicator molecules 104 such that a signal is generated by a first photodetector 224 in response thereto is indicative of the level of fluorescence 331 of the emitted by indicator molecules 104 and, thus, the amount of analyte of interest (e.g., glucose).

In some aspects, a part of the excitation light 329 emitted by the light source 108 may be reflected from the analyte indicator material 117 of an indicator portion (e.g., first indicator portion 600) back into the housing 102 of the sensor 100 as reflection light 333, and the indicator molecules 104 may emit emission light 331 in response to being irradiated with a part of the excitation light 329. In some aspects, the emitted light 331 may have a different wavelength than the wavelength of the excitation light 329. In some aspects, the reflected light 333 and the emitted (e.g., fluoresced) light 331 may be absorbed by the first and second photodetectors 224 and 226, respectively, within the housing 102 of the sensor 100.

In some aspects, each of the one or more photodetectors 224, 226 may be covered by a filter that allows only a certain subset of wavelengths of light to pass through. In some aspects, the one or more filters may be thin glass filters. In some aspects, the one or more filters may be thin film (e.g., dichroic) filters deposited on the glass and may pass only a narrow band of wavelengths and otherwise reflect most of the received light. In some aspects, the filters may be thin film (dichroic) filters deposited directly onto the photo detectors and may pass only a narrow band of wavelengths and otherwise reflect most of the light received thereby.

In some aspects, the filter over the one or more second photodetectors 226 may allow the reflected excitation light 333 to pass through, and the filter over the one or more first photodetectors 224 may allow the emission light 331 to pass through. In some aspects, the one or more second photodetectors 226 may detect an amount of excitation light 333 that is reflected from the analyte indicator material 117. In some aspects, the one or more first photodetectors 224 may detect an amount of emission light 331 that is emitted from the indicator molecules 104 in the analyte indicator material 117 of the sensing area. In some aspects, the peak emission of the indicator molecules 104 may occur around 435 nm, and the one or more first photodetector 224 may be covered by a signal filter that passes light in the range of about 380 nm to 600 nm. In some aspects, higher glucose levels/concentrations correspond to a greater amount of emission light 331 from the indicator molecules 104 in the analyte indicator material 117, and, therefore, a greater number of photons striking the one or more first photodetectors 224.

In some aspects, as shown in FIG. 3A, the sensor 100 may include the housing 102, the circuitry 270, a power source 202, first and second electrically conductive leads 276 and 278, and/or a coupler 324. In some aspects, as shown in FIG. 3B, a first end of the coupler 324 may be attached to the power source 202. In some aspects, as shown in FIG. 3B, when the sensor 100 is assembled, the circuitry 270 may be at least partially within the housing 102. In some aspects, as shown in FIG. 3B, at least a portion of the housing 102 may extend into a second end of the coupler 324.

In some aspects, as shown in FIG. 3A, the circuitry 270 of the sensor 100 may include one or more substrates 112 and one or more circuit components 111. In some aspects, the one or more substrates 112 may be circuit boards (e.g., one or more flexible and/or rigid printed circuit boards (PCBs)). In some aspects, one or more of the circuit components 111 may be mounted or otherwise attached to the one or more substrates 112. However, in some alternative aspects, the one or more substrates 112 may be semiconductor substrates having one or more of the circuit components 111 fabricated therein. For instance, the fabricated circuit components may include analog and/or digital circuitry. Also, in some aspects in which the substrate 112 is a semiconductor substrate, in addition to the one or more circuit components fabricated in the semiconductor substrate, one or more circuit components may be mounted or otherwise attached to the semiconductor substrate. In other words, in some semiconductor substrate aspects, a portion or all of the circuit components 111, which may include discrete circuit elements, an integrated circuit (e.g., an application specific integrated circuit (ASIC)) and/or other electronic components (e.g., a non-volatile memory), may be fabricated in the semiconductor substrate with the remainder of the circuit components 111 secured to the semiconductor substrate, which may provide communication paths between the various secured components.

In some aspects, as shown in FIG. 3A, the one or more light sources 108 and one or more photodetectors 224, 226 of the circuitry 270 may be mounted on and/or fabricated in the one or more substrates 112. In some aspects, as shown in FIG. 3A, the one or more substrates 112 may include (i) a first sensing area with a first set of one or more light sources 108 and one or more photodetectors 224, 226 and (ii) a second sensing area with a second set of one or more light sources 108 and one or more photodetectors 224, 226. In some aspects, the one or more light sources 108 may be mounted on the one or more substrates 112, the one or more photodetectors 224, 226 may be fabricated in the substrate 112, and all or a portion of the circuit components 111 may be fabricated within the substrate 112.

In some aspects, as shown in FIG. 3A, the antenna 114 may be an inductor including a conductor 702 in the form of a coil and a core 704. In some aspects, the core 704 may have a magnetic permeability greater than the magnetic permeability of free space. That is, in some aspects, the core 704 may have a relative magnetic permeability (μᵣ) greater than 1. In some aspects, the core 704 may include, for example and without limitation, ferrite, NiZn, and/or MnZn. However, this is not required, and, in some alternative aspects, different materials may be used for the core 704. In some aspects, the antenna 114 may be, for example, a ferrite-based micro-antenna. In some aspects, as illustrated in FIG. 3A, the one or more substrates 112 of the sensor 100 may be attached to the antenna 114. In some aspects, the circuitry 270 may be connected electrically to the antenna 114. In some aspects, the sensor 100 may use the antenna 114 to communicate data (e.g., measurement data) to the external device 101 and/or the display device 107. In some aspects, the sensor 100 may use the antenna 114 for NFC. However, this is not required, and, in some alternative aspects, the sensor 100 may use a different wireless communication standard.

In some aspects, as shown in FIG. 3A, the circuitry 270 of the sensor 100 may include a PCB 280. In some aspects, the circuitry 270 may include one or more capacitors 282, which may be mounted on the PCB 280. In some aspects, the PCB 280 may include the first and second contact pads 272 and 274. In some aspects, the circuitry 270 (e.g., the circuit components 111) of the substrates 112 and/or the antenna 114 may be connected electrically to the one or more capacitors 282 and/or the first and second contact pads 272 and 274.

In some aspects, the sensor 100 (e.g., the circuitry 270 of the sensor 100) may be powered at least partially by the power source 202. In some aspects, the power source 202 may be an energy storage device (e.g., a battery, fuel cell, capacitor, or super capacitor). In some aspects, at least the exterior of the power source 202 may be made of a biocompatible material such as, for example and without limitation, stainless steel or a titanium alloy. In some aspects, the power source 202 may be a titanium-cased, hermetically-sealed battery. In some aspects, as shown in FIGS. 3A and 3B, the circuitry 270 of the sensor 100 may extend away from the power source 202 along the longitudinal axis of the power source 202.

In some aspects, the power source 202 may include first and second terminals (e.g., a positive terminal (cathode) and a negative terminal (anode)). In some aspects, the first and second electrically conductive leads 276 and 278 may be connected electrically to the first and second terminals, respectively, of the power source 202. In some aspects, the electrically conductive leads 276 and 278 may electrically connect the first and second terminals, respectively, of the power source 202 to the circuitry 270 of the sensor 100. In some aspects, the electrically conductive leads 276 and 278 may be rods or beams including or made out of a conductive material.

In some aspects the sensor 100 may not have an internal power source and may instead be powered in another manner, such as, for example, solely through induction by an external power source (e.g., the external device 101 and/or the display device 107). FIGS. 2, 5A, 5B, 6A, and 6B illustrate the sensor 100 without a power source 202, and FIGS. 3A and 3B illustrate the sensor 100 with a power source.

In some aspects, as shown in FIGS. 3A and 3B, the coupler 324 may be a flange. In some aspects, as shown in FIG. 3B, the coupler 324 may be attached to the power source 202. In some aspects, the coupler 324 may be welded (e.g., laser welded) to the power source 202. In some aspects, the coupler 324 may enclose the first and second terminals of the power source 202. In some aspects, as shown in FIG. 3B, the coupler 324 may be between the housing 102 and the power source 202. In some aspects, as shown in FIG. 3B, the sensor 100 may further include a cap 266 over the one or more openings 268 of the coupler 324.

In some aspects, the coupler 324 may have a generally cylindrical shape. However, other shapes (e.g., a generally rectangular prism shape) may be used in alternative aspects. In some aspects, the coupler 324 may be made of a biocompatible material such as, for example and without limitation, glass, ceramic, stainless steel, titanium, or a titanium alloy. In some aspects, the coupler 324 may include a flat surface that abuts and is attached to the power source 202.

In some aspects, as shown in FIGS. 3A and 3B, the coupler 324 may include one or more openings 268 through which the first and second electrically conductive leads 276 and 278 are capable of being laser welded to the first and second contact pads 272 and 274, respectively, of the circuitry 270. In some aspects, the housing 102 may include one or more openings 103 through which the first and second electrically conductive leads 276 and 278 are capable of being laser welded to the first and second contact pads 272 and 274, respectively, of the circuitry 270.

In some aspects, as shown in FIG. 3B, the sensor 100 may further include an encasement material 109 that encases at least a first portion of the circuitry 270 in the housing 102. In some aspects, the first portion of the circuitry 270 may include the one or more light sources 108 and the one or more photodetectors 224. In some aspects, the encasement material 109 may include a water-resistant epoxy.

In some aspects, the encasement material 109 may be a first encasement material that encases the first portion of the circuitry 270, and the first portion of the circuitry may not include the first and second contact pads 272 and 274. In some aspects, the sensor 100 may further include a second encasement material that encases the first and second electrically conductive leads 276 and 278 and a second portion of the circuitry 270. In some aspects, the second portion of the circuitry may include the first and second contact pads 272 and 274. In some aspects, the first and second encasement materials may be different. In some alternative aspects, the first and second encasement materials may be the same. In some aspects, the second encasement material may include a water-resistant epoxy. In some aspects, the first encasement material may fill a first portion of the housing 102, and the second encasement material may fill the coupler 324 and a second portion of the housing 102 that is not filled by the first encasement material.

In some alternative aspects, instead of first and second encasement materials, the encasement material may include a single encasement material that encases the circuitry 270 and the first and second electrically conductive leads 276 and 278. In some aspects, the encasement material may fill the housing 102 and the coupler 324.

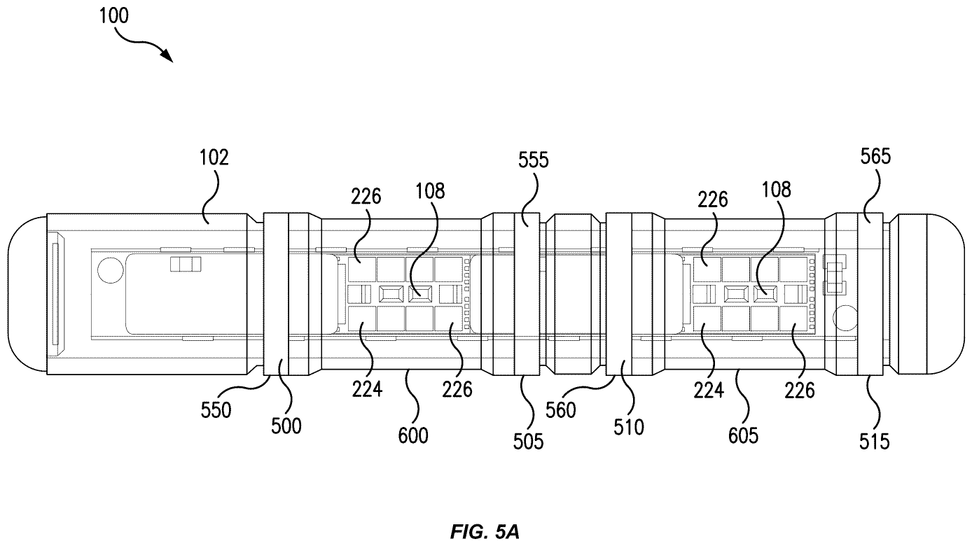

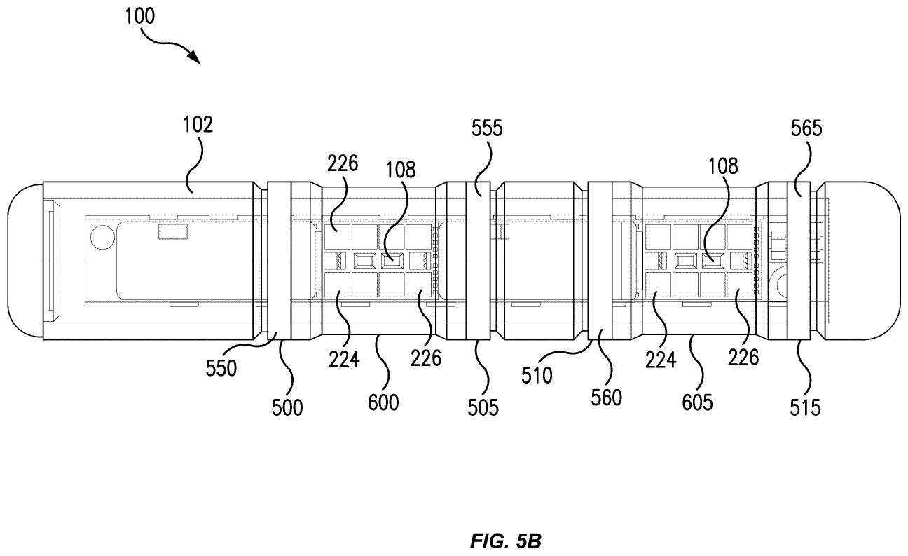

In some aspects, as shown in FIG. 5A, the indicator portions 600 and 605 (and the analyte indicator material 117 on or in the indicator portions 600 and 605) may extend, in a direction of the longitudinal axis of the housing 102, beyond the respective sensing areas of the circuitry 270. However, this is not required, and, in some alternative aspects, as shown in FIG. 5B, the indicator portions 600 and 605 (and the analyte indicator material 117 on or in the indicator portions 600 and 605) may not extend, in a direction of a longitudinal axis of the housing 102, beyond the respective sensing areas of the circuitry 270. That is, in some aspects, the analyte indicator material 117 may be limited to covering only the one or more sensing areas of the circuitry 270 of the sensor 100.

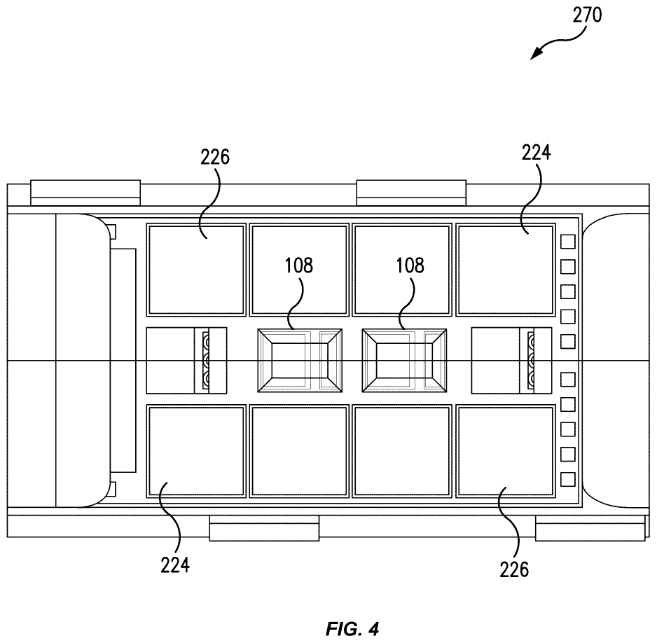

In some aspects, and as shown in FIG. 4, the one or more light sources 108 and one or more photodetectors 224 of a sensing area of the circuitry 270 may be spatially separated. For example, in some aspects, as shown in FIG. 4, each sensing area may include multiple light sources 108 and multiple photodetectors 224, 226. In some aspects, the center of the light sources 108 may be about 0.1 mm, about 0.2 mm, about 0.3 mm, about 0.4 mm, about 0.5 mm, about 0.6 mm, about 0.7 mm, about 0.8 mm, about 0.9 mm, or about 1.0 mm apart. In some aspects, the centers of the first and second light sources 108 may be between about 0.1 and 0.5 mm apart, between about 0.25 and 0.75 mm apart, between about 0.5 and 1.5 mm apart, or between about 1 and about 2.5 mm apart. In such aspects, the centers of a first photodetector 224 and a second photodetector 226 may be about 0.5 mm, about 0.6 mm, about 0.8 mm, about 1.0 mm, about 1.5 mm, about 2.0 mm, about 2.5 mm, about 3.0 mm, about 3.5 mm, or about 5 mm apart. In some aspects, the centers of the photodetector may be between about 0.5 and 1.0 mm apart, between about 1.0 and 2.0 mm apart, between about 1.5 and 3.5 mm apart, or between about 2.0 and about 5.0 mm apart. In some aspects, the centers of the light sources 108 may be about 0.576 mm (or about 0.0227 inches) apart. In such aspects, the centers of the first and second photodetectors 224 and 226 may be about 1.818 mm (or about 0.0716 inches) apart.

In some aspects, the excitation light 329 emitted by the one or more light sources 108 of a sensing area of the circuitry 270 may reach the analyte indicator material 117 of an indicator portion 600 or 605 of the housing 102 after passing through the encasement material (e.g., the first encasement material or the single encasement material). In some aspects, the emission light 331 emitted by the indicator molecules 104 of the analyte indicator material 117 of an indicator portion 600 or 605 of the housing 102 may reach the one or more photodetectors 224 after passing through the encasement material (e.g., the first encasement material or the single encasement material).

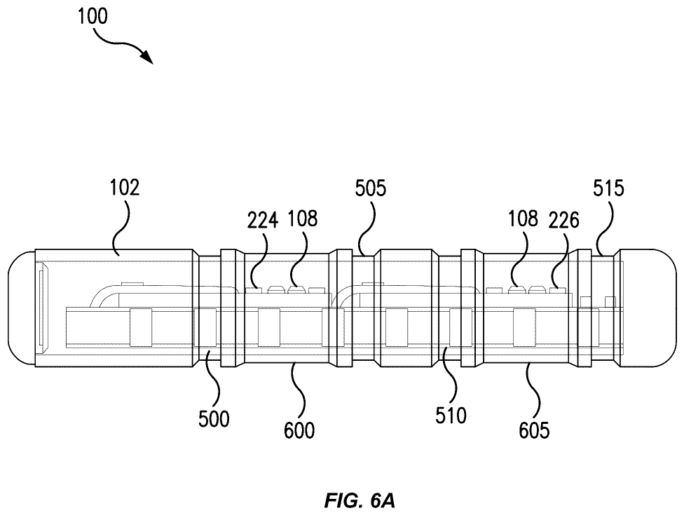

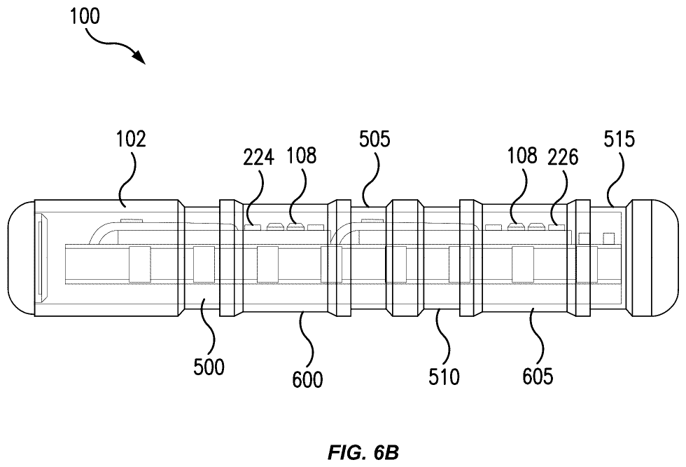

In some aspects, as shown in FIGS. 2, 5A, 5B, 6A, and 6B, the exterior surface of the housing 102 of the sensor 100 may include a first drug portion 500 on a first side of the indicator portion 600 and a second drug portion 505 on a second side of the indicator portion 600 that is opposite the first side of the indicator portion 600. That is, in some aspects, as described longitudinally along the sensor 100, an indicator portion 600 may be bordered on a first side by a first drug portion 500 and on a second side by a second drug portion 505. In some aspects in which the circuitry 270 of the sensor 100 includes first and second sensing areas and in which the housing 102 of the sensor 100 includes first and second indicator portions 600 and 605, as shown in FIGS. 5A, 5B, 6A, and 6B, the exterior surface of the housing 102 of the sensor 100 may include a third drug portion 510 on a first side of the second indicator portion 605 and a fourth drug portion 515 on a second side of the second indicator portion 605 that is opposite the first side of the second indicator portion 605.

In some aspects, the sensor 100 may include drug-eluting material in or on the drug portions of the exterior surface of the housing 102. In some aspects, as shown in FIGS. 2, 5A, and 5B, the sensor 100 may include first drug-eluting material 550 in or on the first drug portion 500 of the exterior surface of the housing 102. In some aspects, as shown in FIGS. 2, 5A, and 5B, the sensor 100 may include second drug-eluting material 555 in or on the second drug portion 505 of the exterior surface of the housing 102. Thus, in some aspects, and as shown in FIGS. 2, the exterior surface of the housing 102 of the sensor 100 may include first and second drug-eluting materials 550 and 555 on opposite sides of the analyte indicator material 117 in or on the first indicator portion 600. In some aspects in which the circuitry 270 of the sensor 100 includes first and second sensing areas and in which the housing 102 of the sensor 100 includes first and second indicator portions 600 and 605, as shown in FIGS. 5A and 5B, the sensor 100 may include third and fourth drug-eluting material 560 and 565 in or on the third and fourth drug portions 510 and 515, respectively, of the exterior surface of the housing 102. Thus, in some aspects, the exterior surface of the housing 102 of the sensor 100 may include third and fourth drug-eluting materials 560 and 565 on opposite sides of the analyte indicator material 117 in or on the second indicator portion 605. In such aspects, the exterior surface of the housing 102 of the sensor 100 may include four drug portions 500, 505, 510, and 515 adjacent to the indicator portions 600 and 605, which may be referred to herein as a “quad” configuration.

In some aspects, the drug-eluting material of the sensor 100 may include one or more therapeutic agents that reduce deterioration of the analyte indicator material 117 of the sensor 100. For example, in some aspects, the first and second drug-eluting material 550 and 555 in or on the first and second drug portions 500 and 505, respectively, of the exterior surface of the housing 102 may each include one or more therapeutic agents that reduce deterioration of the analyte indicator material 117 in or on first indicator portion 600 of the exterior surface of the housing 102. In some aspects in which the circuitry 270 of the sensor 100 includes first and second sensing areas and in which the housing 102 of the sensor 100 includes first and second indicator portions 600 and 605, a third and fourth drug-eluting material 560 and 565 in or on the third and fourth drug portions 510 and 515, respectively, of the exterior surface of the housing 102 may each include one or more therapeutic agents that reduce deterioration of at least the analyte indicator material 117 in or on second indicator portion 605 of the exterior surface of the housing 102.

In some aspects, the one or more therapeutic agents of the drug-eluting material 550, 555, 560, and/or 565 may be incorporated within the drug-eluting material 550, 555, 560, and/or 565 via one or more covalent bonds that break in the presence of aqueous media and release the one or more therapeutic agents of the drug-eluting material 550, 555, 560, and/or 565. In some aspects, the one or more therapeutic agents of the drug-eluting material 550, 555, 560, and/or 565 may be incorporated within the drug-eluting material 550, 555, 560, and/or 565 via one or more covalent bonds that break through exposure to light and release the one or more therapeutic agents of the drug-eluting material 550, 555, 560, and/or 565.

In some aspects, the drug-eluting material 550, 555, 560, and/or 565 may elute the one or more therapeutic agents when one or more of the covalent bonds are broken. For example, in some aspects, the covalent bonds may break in the presence of water (e.g., in the presence of water in the interstitial fluid, blood, or intraperitoneal fluid). However, this is not required, and, in some alternative aspects, the covalent bonds may additionally or alternatively break through exposure to ultraviolet or visible light. In some aspects, the covalent bonds may break through exposure to light emitted by the light source 108. For example, in some aspects, exposure to the excitation light 329 (e.g., having a wavelength of approximately 378 nm) emitted by the light source 108 may cause the covalent bonds to break. Moreover, the light source 108 may be controlled to emit light in a manner (e.g., blinking at specific intervals and/or intensities) that alters (e.g., increases the rate at which one or more therapeutic agents are released) the elution profile of the one or more therapeutic agents (e.g., to maximize effectiveness in preventing oxidation of the indicator species). In some embodiments, a wavelength in a specific range (e.g., 150 nm-1000 nm or 300-600 nm) may be necessary to photocleave (i.e., break the covalent bonds and release), and the wavelength of the light emitted by the light source 108 of the sensor 100 may be within in the specific range.

In some aspects, the one or more therapeutic agents of the drug-eluting material 550, 555, 560, and/or 565 may include an anti-inflammatory drug. In some aspects, the anti-inflammatory drug may be a non-steroidal anti-inflammatory drug (NSAID). In some aspects, the non-steroidal anti-inflammatory drug may be acetylsalicylic acid (aspirin), isobutylphenylpropanoic acid (ibuprofen), naproxen, diclofenac, indomethacin, ketoprofen, ketorolac, meloxicam, and/or celecoxib. In some aspects, the one or more therapeutic agents of the drug-eluting material 550, 555, 560, and/or 565 may include a steroid hormone. In some aspects, the one or more therapeutic agents may include a glucocorticoid. In some aspects, the one or more therapeutic agents may include one or more of dexamethasone, triamcinolone, betamethasone, methylprednisolone, beclometasone, fludrocortisone, derivatives thereof, and analogs thereof. In some aspects, the one or more therapeutic agents may reduce oxidation of the analyte indicator material 117.

In some aspects, the drug-eluting material 550, 555, 560, and/or 565 may elute or release the one or more therapeutic agents in a controlled manner. For instance, in some aspects, the drug-eluting material 550, 555, 560, and/or 565 may release the one or more therapeutic agents in a controlled manner over a period of hours, days, weeks, or months. In some aspects, each drug-eluting material 550, 555, 560, and/or 565 may have different elution/release rates. For example, in some aspects, the first drug-eluting material 550 may release one or more therapeutic agents at a first rate, and the second drug-eluting material 555 may release one or more therapeutic agents at a second rate that is different from the first rate. For example, in some aspects, a faster release rate may be used on the initial immune response (e.g. 0-21 days), and a slower release rate may be used as a maintenance release to moderate any chronic immune response (e.g. 14-365+ days).

In some aspects, chemical degradation and/or oxidation of the indicator molecules 104 of the analyte indicator material 117 may be detrimental to the functioning of the sensor 100 and may result from adverse physiological reactions that may be exhibited by a user/patient's body following implantation or insertion of the sensor 100 into the user/patient's body. The reactions may range from infections due to implantation surgery to the immunological response of a foreign object implanted in the body. That is, the performance of the sensor 100 may be hindered or permanently damaged in vivo via the immunological response to an infection or the sensor 100 itself. In particular, the performance of the indicator molecules 104 of the analyte indicator material 117 may be deteriorated by the immunological response of the body into which the sensor 100 is implanted. For example, white blood cells, including neutrophils, may attack an implanted sensor 100. The neutrophils release, inter alia, hydrogen peroxide, which may degrade indicator molecules 104 (e.g., by oxidizing a boronate group of an indicator molecule 104 and disabling the ability of the indicator molecule 104 to bind glucose). Further, proteins, macrophages, and other types of cells and cellular materials may attach to, react with, or be absorbed by the analyte indicator material 117 leading to immunogenicity, biofouling, and reduced biocompatibility. Further, in some aspects, infection and bacterial colonization on the analyte indicator material 117 may require the implanted sensor 100 to be removed. As noted above, in some aspects, the first, second, third, and/or fourth drug-eluting material each include one or more therapeutic agents that reduce deterioration of the analyte indicator material.

In some aspects, as shown in FIGS. 3A, 3B, 5A, 5B, 6A, and 6B, the housing 102 may include one or more cutouts or recesses. In some aspects, as shown in FIGS. 5A, 5B, 6A, and 6B, the one or more indicator portions 600, 605 and/or the drug portions 500, 505, 510, 515 of the housing 102 may correspond to all or a portion of the cutouts or recesses.

It is understood that, as shown in FIGS. 5A and 5B, different embodiments of the invention have different spacings between the components. For example, in one aspect, the spacing between the center of the first drug eluting material and the center of the first indicator portion may be varied to reach a desired configuration. In another aspect, the spacing between the center of the first drug eluting material and the center of the second drug eluting material may be varied to reach a desired configuration. As described below, variation of these distances may achieve different therapeutic effects.

In some aspects, and as shown in FIG. 5A and FIG. 5B, the distance between the center of a first drug portion and the center of a second drug portion can be calculated and varied depending on the desired configuration of the sensor. In some aspects, the center of a first drug portion and the center of a second drug portion is between about 0.01 and 0.05 inches, between about 0.02 and about 0.08 inches, between about 0.10 and about 0.20 inches, between about 0.10 and about 0.30 inches, between about 0.10 and about 0.50 inches, between about 0.25 and about 0.75 inches, or between about 0.10 and about 1.0 inches. In the aspect shown in FIG. 5A, for example, the distance between the center of a first drug portion and the center of a second drug portion is about 0.197 inches. In the aspect shown in FIG. 5B, for example, the distance between the center of a first drug portion and the center of a second drug portion is about 0.167 inches. In some aspects, the distance between the center of a first drug portion and end of a light source or photodetector is about 0.0590 inches, about 0.0597 inches, about 0.0602 inches. About 0.0609 inches, about 0.0707 inches, about 0.0719 inches, about 0.078 inches, about 0.0782 inches, about 0.1061 inches, about 0.1068 inches, about 1.1073 inches, about 0.108 inches, about 0.1178 inches, about 0.119 inches, about 0.1251 inches, or about 0.1263 inches.

Example 1: The tables and data below compare a “conventional” or “singular drug portion” sensor having a single drug portion on the exterior surface of the housing to a “quad” sensor 100 having the first, second, third, and fourth drug portions 500, 505, 510, 515 on the exterior surface of the housing 102. The single drug portion of the convention sensor has the same area as the areas of the drug portions 500, 505, 510, and 515 combined. The exterior surface of the housing of the conventional sensor has a first indicator portion on one side of the single drug portion and a second indicator portion on an opposite side of the single drug portion.

Two versions of a quad sensor (as shown in FIGS. 5A and 5B, respectively), which differ in the distances between specific components, were tested. Table 1A below shows the relative distances between central and distal positions and the closest two Dex collar segments. Specifically, Table 1A shows (i) the distances of central and distal portions of the indicator portions of the conventional sensor to the single drug portion of the conventional sensor and (ii) for each of the two versions of the quad sensor, the distances of central and distal portions of the indicator portions of the quad sensor to the closest two of the drug portions 500, 505, 510, 515 of the quad sensor 100. In some aspects, relative distances between specific positions within the first and second indicator portions (labeled Central (C1, C2) and Distal (D1, D2) positions) and the two closest drug portions 500, 505, 510, 515 containing drug-eluting material (labeled as Dex1, Dex2, Dex3, Dex4 in the Tables below) are shown in Table 1A.

TABLE 1A

| | C1 - | C1 - | D1 - | D1 - | C2 - | C2 - | D2 - | D2 - | |
|---|---|---|---|---|---|---|---|---|---|
| | Dex1 | Dex2 | Dex1 | Dex2 | Dex3 | Dex4 | Dex3 | Dex4 | Sum |
| Conventional | 0.0758 | 0.0958 | 0.1230 | 0.1430 | 0.1076 | 0.0876 | 0.1547 | 0.1347 | 0.9222 |
| Quad (FIG. 5A) | 0.1251 | 0.0719 | 0.0780 | 0.1190 | 0.0792 | 0.1178 | 0.1263 | 0.0707 | 0.7880 |
| Quad (FIG. 5B) | 0.1061 | 0.0609 | 0.0590 | 0.1080 | 0.0602 | 0.1068 | 0.1073 | 0.0597 | 0.6680 |

The final column (at far right, labeled “Sum”) shows the total sum of the preceding measurements to provide an approximation of the distance (in inches) that the therapeutic agent must elute from the drug-eluting material of the drug portion to reach each of the described positions along the sensor.

In some aspects, the lowest values for each position (Central (C1, C2) and Distal (D1, D2)) and which drug portion, containing drug-eluting material (labeled Dex1, Dex2, Dex3, Dex4) is the closest may be calculated, as shown in Table 1B. Specifically, Table 1B shows the lowest distance values for each position, and which drug portion segment (labeled as Dex1, Dex2, Dex3, Dex4 in the Tables below) is the closest.

TABLE 1B

| | C1 | C1 | D1 | D1 | C2 | C2 | D2 | D2 | |
|---|---|---|---|---|---|---|---|---|---|
| | Closest | Value | Closest | Value | Closest | Value | Closest | Value | Sum |
| Default | Dex1 | 0.0758 | Dex1 | 0.1230 | Dex4 | 0.0876 | Dex4 | 0.1347 | 0.4211 |
| Quad (FIG. 5A) | Dex2 | 0.0719 | Dex1 | 0.0780 | Dex3 | 0.0079 | Dex4 | 0.0707 | 0.2285 |
| Quad (FIG. 5B) | Dex2 | 0.0609 | Dex1 | 0.0590 | Dex3 | 0.0601 | Dex4 | 0.0597 | 0.2397 |

The last column shows the total sum of the preceding measurements to provide an approximation of the distance (in inches) that the therapeutic agent must elute from the drug-eluting material of the drug portion to reach each of the described positions along the sensor.

Example 2: As shown in FIG. 6A, the width of the drug portion may be varied in different embodiments of the invention. That is, in some aspects, the width of the drug portion may be about 0.1, about 0.2, about 0.25, about 0.3, about 0.33, about 0.4, about 0.5, about 0.6, about 0.7, about 0.75, about 0.8, or about 0.9 the width of a drug portion in a configuration where the sensor includes only one drug portion (“default” or “singular drug portion”). In a preferred embodiment, the width of each drug portion may be about 0.25 the width of the singular drug portion. In another preferred embodiment, the width of each drug portion may be about 0.50 the width of the singular drug portion.

In some aspects, and as shown in FIG. 6B, changing the width of each drug portion may further adjust the distance between the circuitry of the sensing area and drug portion. For example, in some aspects, changing the width of each drug portion may change the distance that the therapeutic agent must elute from the drug-eluting material of the drug portion to reach each of the described positions along the sensor, which in term may adjust the therapeutic effect.

TABLE 2

| | LED UV | LEC UV | SEC UV | SED UV |
|---|---|---|---|---|
| | LED | LED | LED | LED |
| Current Dex Distance to close edge of Dex (mm) | 3.15 | 1.33 | 1.51 | 3.33 |
| Current Dex Distance to far edge of Dex (mm) | 5.18 | 3.36 | 3.54 | 5.36 |
| Quad Dex Distance to close edge (mm) | 1.05 | 1.05 | 1.05 | 1.05 |
| Quad Dex Distance to 2^{nd} far edge of Dex (mm) | 3.88 | 3.88 | 3.88 | 3.88 |

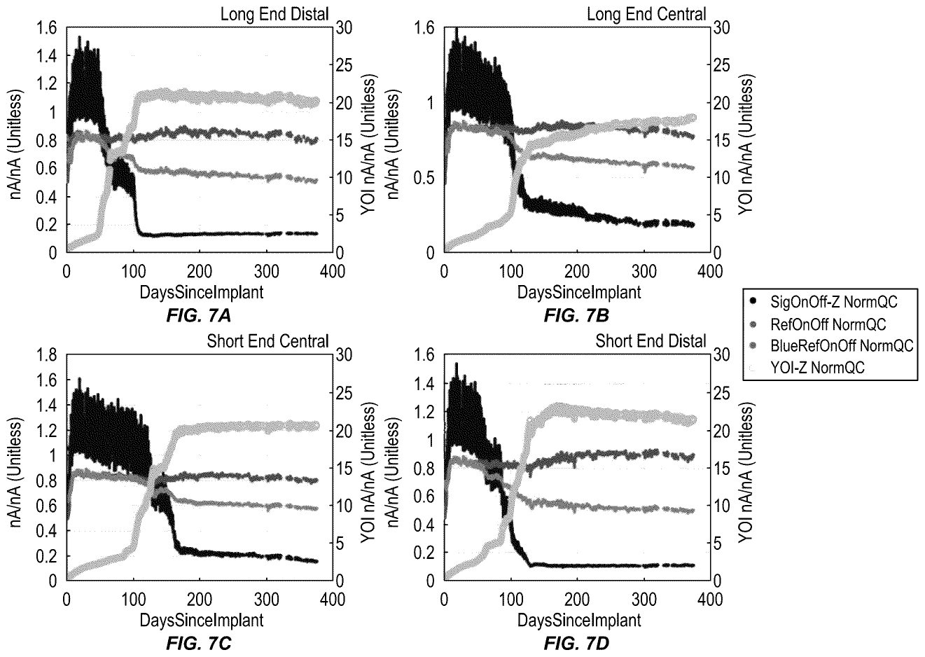

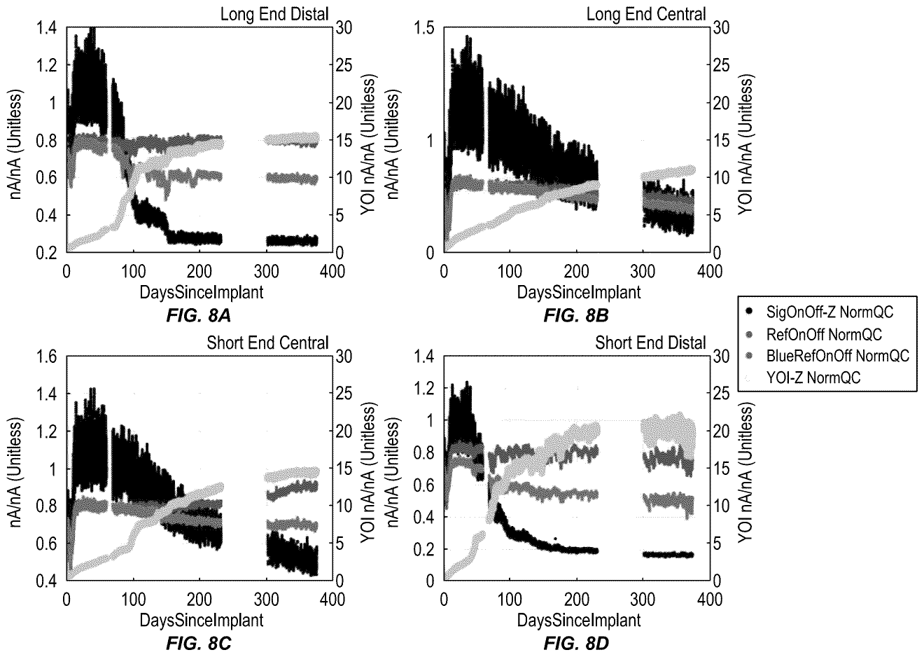

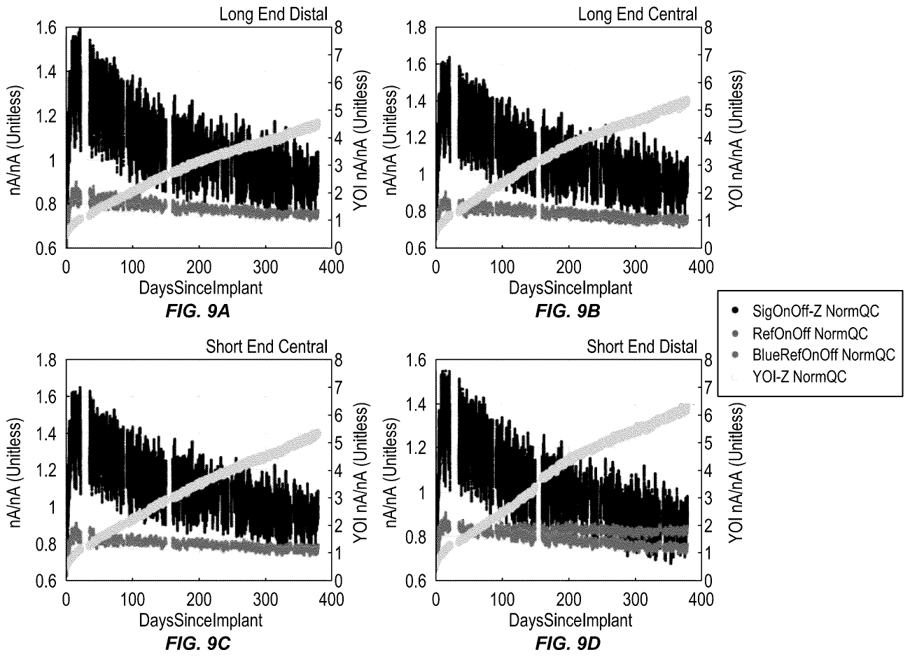

Example 3: As shown in FIGS. 7A-D, 8A-D, and 9A-D, burst oxidation was measured at various locations of the sensor housing. Specifically, burst oxidation was measured at a long end distal portion of analyte indicator material on or in a first indicator portion of a housing of the sensor (FIGS. 7A, 8A, 9A). Burst oxidation was measured at a long end central portion of the analyte indicator material on or in a first indicator portion of a housing of the sensor (FIGS. 7B, 8B, 9B). Burst oxidation was measured at a short end central portion of analyte indicator material on or in a second indicator portion of the housing (FIGS. 7C, 8C, 9C). Finally, burst oxidation was measured at a short end distal portion of the analyte indicator material on or in the second indicator portion of the housing (FIGS. 7D, 8D, 9D). Burst oxidative rates refer to a rapid release of reactive oxygen species (ROS) that can be triggered under certain conditions.

The measurements captured herein, as shown in FIGS. 7A-D, 8A-D, and 9A-D, show where the sensor may be more prone to oxidative damage and, consequently, where drug-eluting material or indicator elements may degrade faster. Thus, calculation of oxidative rates at various points on the sensor allow for optimization of sensor layout and material placement with regards to minimization of ROS effects and potential improvement of sensor durability and performance.

Oxidative burst percentages with regard to sensor configuration are shown in Table 3. The sensors examined had the following configurations: (1) a single sensing area with no sacrificial boronic acid (SBA) in the analyte indicator material and a single drug portion, (2) a single sensing area with SBA in the analyte indicator material a single drug portion, (3) four sensing areas with SBA in the analyte indicator material and single drug portion, and (4) four sensing areas with SBA in the analyte indicator material and four drug portions.

TABLE 3

| Configuration | Sensors (n) | Total | SED | SEC | LEC | LED |
|---|---|---|---|---|---|---|
| 1 (single sensing area, no SBA, | 183 @ Day | 2/183 | N/A | N/A | N/A | N/A |
| single drug portion) | 180 | (1%) | | | | |
| 2 (single sensing area, SBA, | 43 @ Day | 2/43 | N/A | N/A | N/A | N/A |
| single drug portion) | 180 | (4.7%) | | | | |
| 3 (four sensing areas, SBA, | 16/16 @ | 16/64 | 6/16 | 1/16 | 2/16 | 7/16 |
| single drug portion) | Day 360 | (25%) | (37.5%) | (6.25%) | (12.5%) | (43.75%) |
| 4 (four sensing areas, SBA, | 16/16 @ | 0/64 | 0/16 | 0/16 | 0/16 | 0/16 |
| four drug portions) | Day 360 | (0%) | (0%) | (0%) | (0%) | (0%) |

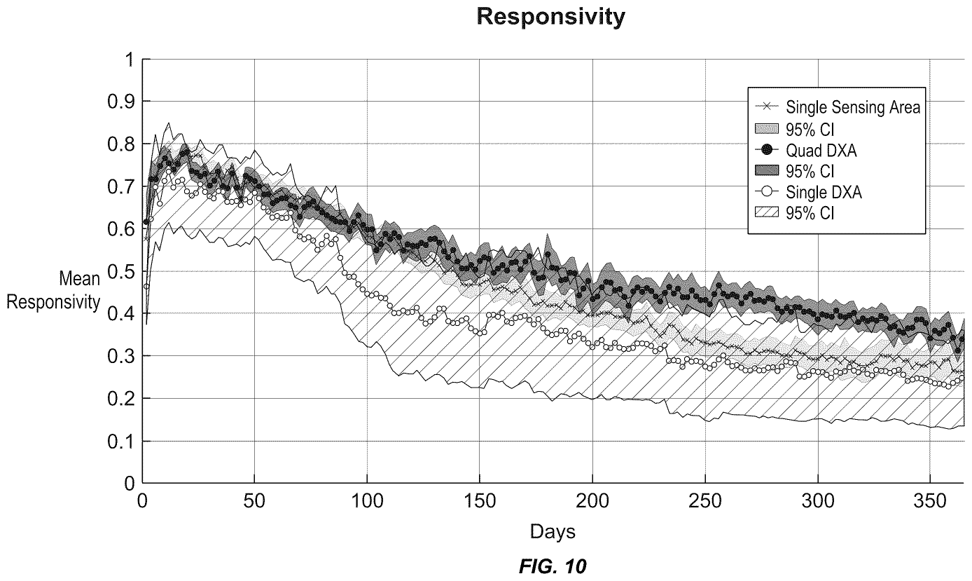

Example 4: As shown in FIG. 10, mean responsivity of three variations of a sensor was examined over time from 0 and 350 days. The sensors examined were (i) a configuration with a single sensing area and a single drug portion (responsivity shown with Xs and light gray shading, referred to as “Single Sensing Area”), (ii) a configuration with four sensing areas and a single drug portion configuration (responsivity shown with Os and crosshatching, referred to as “Single DXA”), and (iii) a configuration with four sensing areas and four drug portions (responsivity shown with dots and dark gray shading, referred to as “Quad DXA”).

Results for the responsivity experiment are shown in FIG. 10. These comparisons suggest that a “quad” sensor with drug portions on opposite sides of each indicator portion may significantly improve responsivity and reduce deterioration of analyte indicator material in or on the indicator portions.

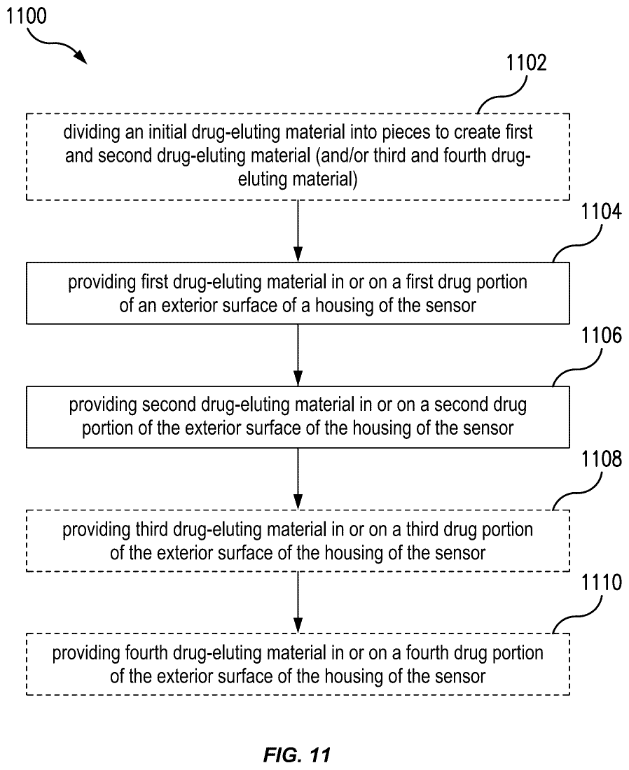

FIG. 11 is a flowchart illustrating a process 1100 for manufacturing the sensor 100 for measurement of an analyte in a medium within a living animal.

In some aspects, the first and second drug-eluting material 550 and 555 may be pieces of an initial drug-eluting material. In some aspects, the third and fourth drug-eluting material 560 and 565 may also be pieces of the initial drug-eluting material. In some aspects, the initial drug-eluting material may have been previously tested or validated to confirm that the initial drug-eluting material has the one or more therapeutic agents, which reduce deterioration of the analyte indicator material 117, in an amount above a minimum threshold and/or within a predetermined range (e.g., 0.1 μg/day to 20 μg/day, 0.5 μg/day to 12 μg/day, 1 μg/day to 6 μg/day, 1.2 μg/day to 5.5 μg/day, or 2 μg/day to 4 μg/day). In some aspects, the initial drug-eluting material may have been previously approved by a regulatory agency (e.g., the Food and Drug Administration (FDA), the European Medicines Agency (EMA), or a Notified Body) to be safe and effective for ameliorating immune response to an implantable device (e.g., a sensor 100) in a living animal (e.g., a human). In some aspects, the initial drug-eluting material may additionally or alternatively have been part of an implantable device (e.g., an analyte sensor) that was previously approved by a regulatory agency (e.g., the FDA, EMA, or a Notified Body) for use in a living animal (e.g., a human) with the initial drug-eluting material being safe and effective for ameliorating immune response to the implantable device in the living animal. In some aspects, the process 1100 may include an optional step of 1102 in which the method includes dividing (e.g., cutting, separating, tearing, or pulling apart) the initial drug-eluting material into pieces to create the first and second drug-eluting material 550 and 555 and/or the third and fourth drug-eluting material 560 and 565.

In some aspects, the process 1100 may include a step 1104 of providing the first drug-eluting material 550 in or on the first drug portion 500 of the exterior surface of the housing 102 of the sensor 100. In some aspects, the process 1100 may include a step 1106 of providing the second drug-eluting material 555 in or on the second drug portion 505 of the exterior surface of the housing 102. In some aspects, the first and second drug-eluting material 550 and 555 may each include one or more therapeutic agents that reduce deterioration of the analyte indicator material 117.

In some aspects, the process 1100 may include a step 1108 of providing the third drug-eluting material 560 in or on the third drug portion 510 of the exterior surface of the housing 102 of the sensor 100. In some aspects, the process 1100 may include a step 1110 of providing the fourth drug-eluting material 565 in or on the fourth drug portion 515 of the exterior surface of the housing 102. In some aspects, the third and fourth drug-eluting material 560 and 565 may each include one or more therapeutic agents that reduce deterioration of the analyte indicator material.

Aspects of the present invention have been fully described above with reference to the figures. Although the invention has been described based upon these preferred aspects, it would be apparent to those of skill in the art that certain modifications, variations, and alternative constructions could be made to the described aspects within the spirit and scope of the invention.

Embodiments of the present invention have been fully described above with reference to the drawing figures. Although the invention has been described based upon these preferred embodiments, it would be apparent to those of skill in the art that certain modifications, variations, and alternative constructions could be made to the described embodiments within the spirit and scope of the invention. For example, although in some embodiments, the analyte sensor 100 may be an optical sensor, this is not required, and, in one or more alternative embodiments, the analyte sensor may be a different type of analyte sensor, such as, for example, an electrochemical sensor, a diffusion sensor, or a pressure sensor. Also, although in some embodiments, the analyte sensor 100 may be an implantable sensor, this is not required, and, in some alternative embodiments, the analyte sensor may be a transcutaneous sensor having a wired connection to an external transceiver. For example, in some alternative embodiments, the analyte sensor 100 may be located in or on a transcutaneous needle (e.g., at the tip thereof). In these embodiments, instead of wirelessly communication using an antenna 114 (e.g., inductor), the analyte sensor may communicate with the external transceiver using one or more wires connected between the external transceiver and a transceiver transcutaneous needle including the analyte sensor. For another example, in some alternative embodiments, the analyte sensor may be located in a catheter (e.g., for intravenous blood glucose monitoring) and may communicate (wirelessly or using wires) with an external transceiver.

Additionally, while the process 1100 described above and illustrated in FIG. 11 is shown as a sequence of steps, this was done solely for the sake of illustration. Accordingly, it is contemplated that some steps may be added, some steps may be omitted, the order of the steps may be re-arranged, and some steps may be performed in parallel. For example, although FIG. 11 shows the step 704 being performed before step 706, this is not required, and, in some alternative aspects, step 704 may be performed after step 706 or in parallel with step 706. Similarly, although FIG. 11 shows the step 708 being performed before step 710, this is not required, and, in some alternative aspects, step 708 may be performed after step 710 or in parallel with step 710. Also, although FIG. 11 shows the steps 704 and 706 being performed before steps 708 and 710, this is not required, and, in some alternative aspects, steps 704 and 706 may be performed after steps 708 and 710 or in parallel with steps 708 and 710.

What is claimed is:

1. A sensor for measurement of an analyte in a medium within a living animal, the sensor comprising:
a housing comprising an exterior surface, wherein the exterior surface of the housing comprises an indicator portion, a first drug portion on a first side of the indicator portion, and a second drug portion on a second side of the indicator portion that is opposite the first side of the indicator portion;
circuitry within the housing, wherein the circuitry comprises a sensing area that includes one or more light sources and one or more photodetectors, wherein the indicator portion of the housing corresponds to the sensing area of the circuitry;
analyte indicator material on or in only the indicator portion of the exterior surface of the housing, wherein:
the analyte indicator material comprises indicator molecules configured to reversibly bind the analyte, and the indicator molecules have a detectable property indicative of whether the analyte is bound or unbound;
the one or more lights sources of the sensing area are configured to emit light to the analyte indicator material; and
the one or more photodetectors of the sensing area are configured to receive light reflected from or emitted by the analyte indicator material;
first drug-eluting material in or on the first drug portion of the exterior surface of the housing;
second drug-eluting material in or on the second drug portion of the exterior surface of the housing, wherein the first and second drug-eluting material each comprise one or more therapeutic agents that reduce deterioration of the analyte indicator material.

2. The sensor of claim 1, wherein the sensor is implantable within a living animal.

3. The sensor of claim 1, wherein the analyte indicator material does not extend, in a direction of a longitudinal axis of the housing, beyond the sensing area of the circuitry.

4. The sensor of claim 1, wherein:
the indicator portion of the housing is a first indicator portion, the sensing area is a first sensing area, and the analyte indicator material is first analyte indicator material;
the exterior surface of the housing further comprises a second indicator portion, a third drug portion on a first side of the second indicator portion, and a fourth drug portion on a second side of the second indicator portion that is opposite the first side of the second indicator portion;
the circuitry further comprises a second sensing area that includes one or more light sources and one or more photodetectors, and the second indicator portion of the housing corresponds to the second sensing area of the circuitry; and
the sensor further comprises:
second analyte indicator material on or in only the second indicator portion of the exterior surface of the housing, wherein:
the second analyte indicator material comprises indicator molecules configured to reversibly bind the analyte, and the indicator molecules of the second analyte indicator material have a detectable property indicative of whether the analyte is bound or unbound;
the one or more lights sources of the second sensing area are configured to emit light to the second analyte indicator material; and
the one or more photodetectors of the second sensing area are configured to receive light reflected from or emitted by the second analyte indicator material;
third drug-eluting material in or on the third drug portion of the exterior surface of the housing; and
fourth drug-eluting material in or on the fourth drug portion of the exterior surface of the housing, wherein the third and fourth drug-eluting material each comprise one or more therapeutic agents that reduce deterioration of at least the second analyte indicator material.

5. The sensor of claim 1, wherein the one or more therapeutic agents of the first and second drug-eluting material are incorporated within the first and second drug-eluting material via one or more covalent bonds that break in the presence of aqueous media and release the one or more therapeutic agents of the first and second drug-eluting material.

6. The sensor of claim 1, wherein the one or more of the therapeutic agents of the first and second drug-eluting material are incorporated within the first and second drug-eluting material via one or more covalent bonds that break through exposure to light and release the one or more therapeutic agents of the first and second drug-eluting material.

7. The sensor of claim 1, wherein the one or more therapeutic agents of the first and second drug-eluting material include an anti-inflammatory drug.

8. The sensor of claim 7, wherein the anti-inflammatory drug is a non-steroidal anti-inflammatory drug.

9. The sensor of claim 8, wherein the non-steroidal anti-inflammatory drug is acetylsalicylic acid.

10. The sensor of claim 8, wherein the non-steroidal anti-inflammatory drug is isobutylphenylpropanoic acid.

11. The sensor of claim 1, wherein the one or more therapeutic agents of the first and second drug-eluting material include a glucocorticoid.

12. The sensor of claim 1, wherein the one or more therapeutic agents of the first and second drug-eluting material include one or more of dexamethasone, triamcinolone, betamethasone, methylprednisolone, beclometasone, fludrocortisone, derivatives thereof, and analogs thereof.

13. The sensor of claim 1, wherein the one or more therapeutic agents of the first and second drug-eluting material reduce oxidation of the analyte indicator.

14. A method of manufacturing a sensor for measurement of an analyte in a medium within a living animal, the method comprising:
providing first drug-eluting material in or on a first drug portion of an exterior surface of a housing of the sensor, wherein:
the exterior surface of the housing comprises an indicator portion, the first drug portion on a first side of the indicator portion, and a second drug portion on a second side of the indicator portion that is opposite the first side of the indicator portion;
the sensor includes circuitry within the housing, the circuitry comprises a sensing area that includes one or more light sources and one or more photodetectors, and the indicator portion of the housing corresponds to the sensing area of the circuitry;
the sensor includes analyte indicator material on or in only the indicator portion of the exterior surface of the housing;
the analyte indicator material comprises indicator molecules configured to reversibly bind the analyte, and the indicator molecules have a detectable property indicative of whether the analyte is bound or unbound;
the one or more lights sources of the sensing area are configured to emit light to the analyte indicator material; and
the one or more photodetectors of the sensing area are configured to receive light reflected from or emitted by the analyte indicator material; and
providing second drug-eluting material in or on the second drug portion of the exterior surface of the housing, wherein the first and second drug-eluting material each comprise one or more therapeutic agents that reduce deterioration of the analyte indicator material.

15. The method of claim 14, wherein the sensor is implantable within a living animal.

16. The method of claim 14, wherein the analyte indicator material does not extend, in a direction of a longitudinal axis of the housing, beyond the sensing area of the circuitry.

17. The method of claim 14, wherein:
the indicator portion of the housing is a first indicator portion, the sensing area is a first sensing area, and the analyte indicator material is first analyte indicator material;
the exterior surface of the housing further comprises a second indicator portion, a third drug portion on a first side of the second indicator portion, and a fourth drug portion on a second side of the second indicator portion that is opposite the first side of the second indicator portion;
the circuitry further comprises a second sensing area that includes one or more light sources and one or more photodetectors, and the second indicator portion of the housing corresponds to the second sensing area of the circuitry; and
the sensor further includes second analyte indicator material on or in only the second indicator portion of the exterior surface of the housing;
the second analyte indicator material comprises indicator molecules configured to reversibly bind the analyte, and the indicator molecules of the second analyte indicator material have a detectable property indicative of whether the analyte is bound or unbound;
the one or more lights sources of the second sensing area are configured to emit light to the second analyte indicator material; and
the one or more photodetectors of the second sensing area are configured to receive light reflected from or emitted by the second analyte indicator material;
the method further comprises providing third drug-eluting material in or on the third drug portion of the exterior surface of the housing;
the method further comprises providing fourth drug-eluting material in or on the fourth drug portion of the exterior surface of the housing; and
the third and fourth drug-eluting material each comprise one or more therapeutic agents that reduce deterioration of at least the second analyte indicator material.

18. The method of claim 17, wherein the first, second, third, and fourth drug-eluting material are pieces of an initial drug-eluting material, and the method further comprises dividing the initial drug-eluting material into pieces to create the first, second, third, and fourth drug-eluting material.

19. The method of claim 14, wherein the one or more therapeutic agents of the first and second drug-eluting material are incorporated within the first and second drug-eluting material via one or more covalent bonds that break in the presence of aqueous media and release the one or more therapeutic agents of the first and second drug-eluting material.

20. The method of claim 14, wherein the one or more of the therapeutic agents of the first and second drug-eluting material are incorporated within the first and second drug-eluting material via one or more covalent bonds that break through exposure to light and release the one or more therapeutic agents of the first and second drug-eluting material.

21. The method of claim 14, wherein the one or more therapeutic agents of the first and second drug-eluting material include an anti-inflammatory drug.

22. The method of claim 21, wherein the anti-inflammatory drug is a non-steroidal anti-inflammatory drug.

23. The method of claim 14, wherein the one or more therapeutic agents of the first and second drug-eluting material include a glucocorticoid.

24. The method of claim 14, wherein the one or more therapeutic agents of the first and second drug-eluting material include one or more of dexamethasone, triamcinolone, betamethasone, methylprednisolone, beclometasone, fludrocortisone, derivatives thereof, and analogs thereof.

25. The method of claim 14, wherein the one or more therapeutic agents of the first and second drug-eluting material reduce oxidation of the analyte indicator.

26. The method of claim 14, the first drug-eluting material and the second drug-eluting material are pieces of an initial drug-eluting material, and the method further comprises dividing the initial drug-eluting material into pieces to create the first drug-eluting material and the second drug-eluting material.
